(19) United States
(12) Patent Application Publication
(10) Pub. No.: US 2006/0257495 A1
(43) Pub. Date: Nov. 16, 2006

(54) Method of purification of polyalkylene materials

(75) Inventors: San-Ming Yang, Mississauga (CA); Thomas E. Enright, Tottenham (CA); Aurelian Veleriu Magdalinis, Aurora (CA); Ahmed Abd Alzamly, Mississauga (CA); Man-Chung Tam, Mississauga (CA); Carol Ann Jennings, Toronto (CA); Peter M. Kazmaier, Mississauga (CA); Marko D. Saban, Etobicoke (CA)

Correspondence Address: FAY, SHARPE, FAGAN, MINNICH & MCKEE, LLP, 1100 SUPERIOR AVENUE, SEVENTH FLOOR, CLEVELAND, OH, 44114, US

(73) Assignee: XEROX CORPORATION

(21) Appl. No.: 11/126,745

(22) Filed: May 11, 2005

(51) Int. Cl. G03G 9/00; A61K 9/50

(52) U.S. Cl. 424/490; 430/108.8

(57) ABSTRACT

The disclosure provides, in various embodiments, a method of purifying polyalkylene. Also included are microencapsulated Gyricon beads, phase change ink, and toners comprising the purified polyalkylene.

### BACKGROUND

The present disclosure is generally directed to various embodiments of a method of purifying or separating polyalkylene materials. The present disclosure also relates to the microencapsulated Gyricon or bichromal beads or balls produced utilizing the purified polyalkylene, as well as phase change inks and toners comprising the same.

Polyalkylene wax such as linear polyethylene wax is a major component used in cyber toners, solid inks, EA toners, and other marking materials. Wax properties such as purity, molecular weight distribution, polydispersity, jetting, and fusion etc. are important for the performances of these applications.

For example, high molecular weight (Mw) wax is used in Gyricon devices, which are utilized in electronic signage. It is found that contrast ratio of Gyricon devices can be increased if purified polyethylene wax is used in the cyber toner formulation.

In this regard, bichromal balls, or beads as sometimes referred to in the art, are tiny spherical balls, such as micron-sized wax beads, which have an optical and an electrical anisotropy. These characteristics generally result from each hemisphere surface or side having a different color, such as black on one side and white on the other, and electrical charge, i.e., positive or negative. Depending on the electrical field produced, the orientation of these beads will change, showing a different color (such as black or white) and collectively create a visual image.

The spherical particles are generally embedded in a solid substrate with a slight space between each ball. The substrate is then filled with a liquid (such as an oil) so that the balls are free to rotate in a changing electrical field, but can not migrate from one location to another. If one hemisphere is black and the other is white. Each pixel can be turned on and off by the electrical field applied to that location. Furthermore, each pixel can be individually addressed, and a full page image can thus be generated.

For example, reusable signage or displays can be produced by incorporating the tiny bichromal beads in a substrate such as sandwiched between thin sheets of a flexible elastomer and suspended in an emulsion. The beads reside in their own cavities within the flexible sheets of material. Under the influence of a voltage applied to the surface, the beads will rotate to present one side or the other to the viewer to create an image. The image stays in place until a new voltage pattern is applied using software, which erases the previous image and generates a new one. This results in a reusable signage or display that is electronically writable and erasable.

Furthermore, electronic displays produced by these bichromal balls or beads are sometimes referred to as “gyricon” displays. This terminology is reportedly the result of a combination of the Greek word for “rotating” and the Latin word for “image.”

Numerous patents describe bichromal balls, their manufacture, incorporation in display systems or substrates, and related uses and applications. Exemplary patents include, but are not limited to: U.S. Pat. Nos. 5,262,098; 5,344,594; 5,604,027 reissued as Re. 37,085; U.S. Pat. Nos. 5,708,525; 5,717,514; 5,739,801; 5,754,332; 5,815,306; 5,900,192; 5,976,428; 6,054,071; 5,989,629; 6,235,395; 6,419,982; 6,235,395; 6,419,982; 6,445,490; and 6,703,074; all of which are hereby incorporated by reference. In addition, disclosure is provided by U.S. Pat. Nos. 4,126,854; and 5,825,529; and N. K. Sheridon et al., “The Gyricon—A twisting ball display”, Proc. SID, Boston, Mass., 289, 1977; T. Pham et al., “Electro-optical characteristics of the Gyricon display”, SID '02 Digest, 199, 2002; which again are hereby incorporated by reference.

However, some commercially supplied polyalkylene waxes fail to meet one or more of the requirements for wax properties. For example, wax products have large batch-to-batch variation, high polydispersity index (PDI), and skewness in Mw distribution etc. These material defects create inconsistent results in the marking products. Sometimes, the wax properties variation is mainly due to the presence of low Mw wax fraction. Moreover, there is no large scale method available to purify wax material.

The disclosure provides a solution that can solve one or more of the aforementioned problems.

### BRIEF DESCRIPTION

In one exemplary embodiment, a method of polyalkylene purification or separation is provided. The method comprises:

(i) providing a polyalkylene with a weight average molecular weight M_{w};

(ii) mixing the polyalkylene with a C₅₋₁₆ alkane;

(iii) dissolving a first portion of the polyalkylene with a weight average molecular weight M_{w1}<M_{w} in the C₅₋₁₆ alkane;

(iv) separating a second portion of the polyalkylene with a weight average molecular weight M_{w2}>M_{w} that is insoluble in the C₅₋₁₆ alkane; and

(v) optionally recovering the first portion of the polyalkylene from its C₅₋₁₆ alkane solution.

In another exemplary embodiment, a microencapsulated Gyricon bead comprising the purified polyalkylene from the above method is provided.

In still another exemplary embodiment, a phase change ink comprising the purified polyalkylene from the above method is provided.

In a further exemplary embodiment, a toner comprising the purified polyalkylene from the above method is provided.

These and other embodiments will be more particularly described with regard to the drawings and detailed description set forth below.

### BRIEF DESCRIPTION OF THE DRAWINGS

The following is a brief description of the drawings, which are presented for the purposes of illustrating one or more of the exemplary embodiments disclosed herein and not for the purposes of limiting the same.

FIG. 1 shows the DSC Analysis of separated polyethylene samples according to one embodiment of the present disclosure.

FIG. 2 shows the HT-GPC statistical analysis of several separated and unseparated polyethylene samples according to one embodiment of the present disclosure.

### DETAILED DESCRIPTION

The disclosure also provides a method of polyalkylene wax separation comprising:

(i) providing a polyalkylene with a weight average molecular weight M_{w}; (ii) mixing the polyalkylene with a C₅₋₁₆ alkane;

(iii) dissolving a first portion of the polyalkylene with a weight average molecular weight M_{w1}<M_{w} in the C₅₋₁₆ alkane;

(iv) separating a second portion of the polyalkylene with a weight average molecular weight M_{w2}>M_{w} that is insoluble in the C₅₋₁₆ alkane; and

(v) optionally recovering the first portion of the polyalkylene from its C₅₋₁₆ alkane solution.

Solvent extraction technique may be employed in the present separation method of polyalkylene such as polyethylene. The term “solvent extraction” herein means the process of transferring a substance from any matrix to an appropriate liquid phase. For example, the polyalkylene with a weight average molecular weight M_{w} (hereinafter “the polyalkylene with M_{w}”) in the method may serve as the “any matrix” or “solid phase”; and a C₅₋₁₆ alkane may serve as the appropriate liquid phase. In the separation process, the first portion of the polyalkylene may be substantially transferred or extracted into the C₅₋₁₆ alkane phase, while the second portion of the polyalkylene can substantially not. Sometimes, commonly known leaching techniques may also be employed in the present method.

In embodiments, the polyalkylene of the disclosure is also commonly called polyalkylene wax, which may be selected from polyethylene wax, polypropylene wax, mixture thereof, and any form of ethylene-propylene copolymer wax. In typical embodiments of the invention, the polyalkylene wax comprises polypropylene wax.

The term “polyethylene” used in the disclosure should not be limitedly understood as a polymer prepared from ethylene. Rather the polyethylene of the disclosure should be understood from a structural point of view. In a sense, the polyethylene typically covers any branched or linear solid alkane with a weight average molecular weight M_{w}. To make this point clear, a polyethylene may be the polymerization products of the following reactions, although the products of chain-growth condensation (1) is called polymethylene to distinguish it from the commercial polymer prepared from ethylene as in (2).

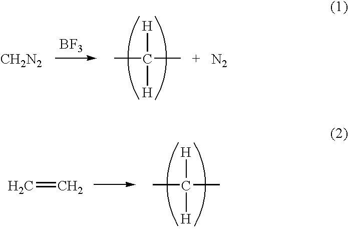

Polyethylene (PE) waxes may be made from ethylene produced from natural gas or by cracking petroleum naphtha. Ethylene may then be polymerized to produce waxes with various melt points, hardnesses and densities etc.

Polyethylene sometimes known as polythene, which is also within the scope of this disclosure. The polyethylene with M_{w} may comprise branched polyethylene, linear polyethylene, or mixture thereof. In typical embodiments, the polyethylene with M_{w} comprises linear polyethylene.

Commercially available polyethylene wax may be obtained under the trade name of Polywax family from Baker-Petrolite, AC PE wax from Honeywell, Licowax PE family from Clariant, Synthetic wax from Salsowax, and Luwax from BASF.

In various embodiments, the value of M_{w} may broadly range from about 425 to about 3,700 such as from about 425 to about 3,000, generally from about 1,700 to about 3,700, typically from about 2,200 to about 3,200, and more typically from about 2,700 to about 2,800. In a specific embodiment of the disclosure, the value of M_{w} is in the neighborhood of 2,740.

It is to be understood herein, that if a “range” or “group” is mentioned with respect to a particular characteristic of the present disclosure, for example, molecular weight, chemical species, and temperature etc., it relates to and explicitly incorporates herein each and every specific member and combination of sub-ranges or sub-groups therein whatsoever. Thus, any specified range or group is to be understood as a shorthand way of referring to each and every member of a range or group individually as well as each and every possible sub-ranges or sub-groups encompassed therein; and similarly with respect to any sub-ranges or sub-groups therein.

According to the disclosure, the polyethylene with M_{w} may be separated into at least two portions. The first portion polyethylene has a weight average molecular, M_{w1}, and is abbreviated herein as “the first portion polyethylene with M_{w1}”; the second portion polyethylene has a weight average molecular, M_{w2}, and is abbreviated herein as “the second portion polyethylene with M_{w2}”. In typical embodiments, separation of the first portion polyethylene and the second portion polyethylene is accomplished based on their solubility difference in the C₅₋₁₆alkane.

The separation method of the disclosure may sometimes be commonly called purification. However, when the terms such as “purify”, “purification”, “purified”, and the like are used, the method should not be understood as to give only purified product and impurities. Depending on what the target product is, the first portion polyethylene with M_{w1}, the second portion polyethylene with M_{w2}, or both, may be commonly and conveniently called purified polyethylene wax such as purified Polywax, or purified Polywax 2000.

In typical embodiments, the second portion polyethylene with M_{w2} is the target product, and is therefore called purified product of the method in those embodiments.

The M_{w1} value of the first portion polyethylene may generally range from about 0.55M_{w} to about 0.95M_{w}, and typically range from about 0.70M_{w} to about 0.75M_{w}. In a specific embodiment, M_{w1}≈0.73M_{w}. For example, M_{w}≈2,746 and M_{w1}≈1,999.

The M_{w2} value of the second portion polyethylene may generally range from about 1.05M_{w} to about 1.45M_{w}, and typically range from about 1.20M_{w} to about 1.30M_{w}. In a specific embodiment, M_{w2}≈1.24M_{w}. For example, M_{w}≈2,746 and M_{w2}≈3,418.

As a skilled artisan understands, polydispersity index (PDI) of polymer is defined as M_{w}/Mₙ, in which Mn is the number average molecular weight of the polymer and Mw is the number average molecular weight of the polymer. In typical embodiments, the polyalkylene such as polyethylene with M_{w} has a polydispersity index PDI; the first portion polyalkylene such as polyethylene with M_{w1} has a polydispersity index PDI₁ which is less than PDI (i.e. PDI₁<PDI); and the second portion polyalkylene such as polyethylene with M_{w2} has a polydispersity index PDI₂ which is also less than PDI (i.e. PDI₂<PDI). As such, the embodiment may be a method of polyalkylene wax separation comprising:

(i) providing a polyalkylene with a weight average molecular weight M_{w} and a polydispersity index PDI;

(ii) mixing the polyalkylene with a C₅₋₁₆ alkane;

(iii) dissolving a first portion of the polyalkylene with a weight average molecular weight M_{w1}<M_{w} and with a polydispersity index PDI₁<PDI in the C₅₋₁₆ alkane;

(iv) separating a second portion of the polyalkylene with a weight average molecular weight M_{w2}>M_{w} and with a polydispersity index PDI₂<PDI that is insoluble in the C₅₋₁₆ alkane; and

(v) optionally recovering the first portion of the polyalkylene from its C₅₋₁₆ alkane solution.

In various embodiments, PDI of the polyalkylene such as polyethylene with M_{w} may generally range from about 1.3 to about 2.0, and both PDI₁ and PDI₂ are in the range of from about 0.78PDI to about 0.98PDI. In a specific embodiment, PDI≈1.45, PDI₁≈1.28, and PDI₂≈1.27.

The C₅₋₁₆ alkane used in the method of this disclosure means acyclic branched or unbranched hydrocarbons having the general formula CₙH₂ₙ₊₂, in which n is an integral number and 5≦n≦16. The C₅₋₁₆ alkane may comprise a normal (n-) alkane, an isomeric (iso-) alkane, or mixture thereof.

In typical embodiments, the C₅₋₁₆ alkane may comprise an isomeric alkane. In more typical embodiments, the C₅₋₁₆ alkane comprises a C₇₋₁₀ isomeric alkane.

Exemplary C₅₋₁₆ alkane may be selected from one of the following compounds or mixture thereof:

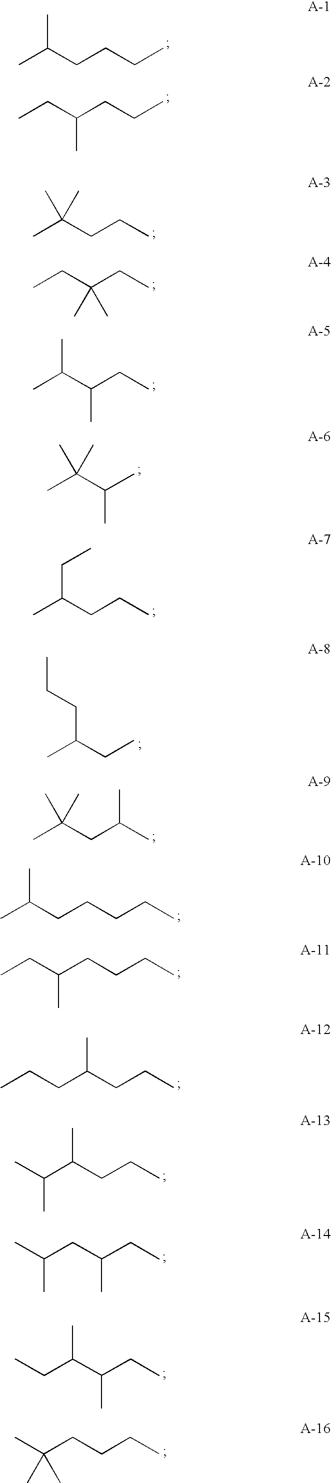

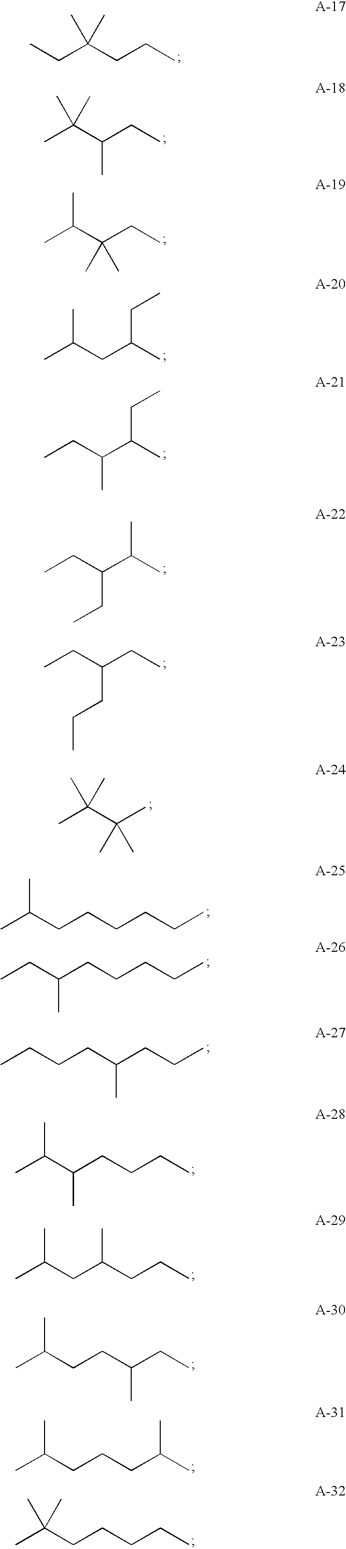

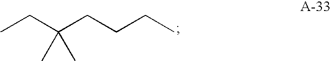

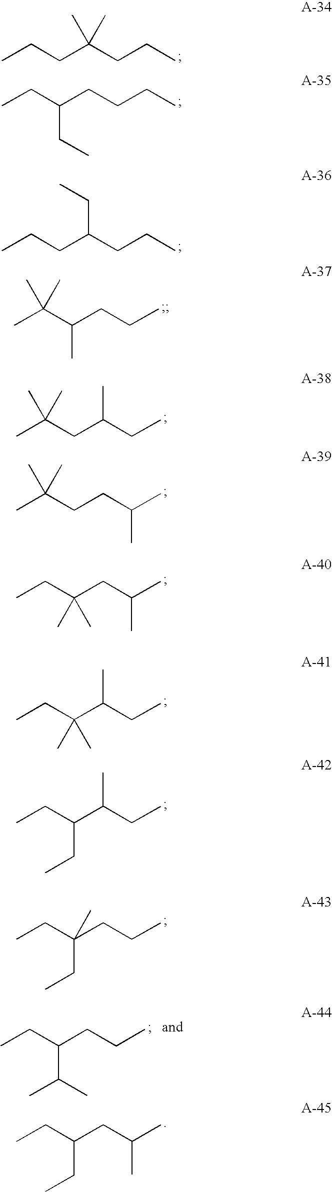

In a specific embodiment, the C₅₋₁₆ alkane comprises the compound having Formula A-9, 2,2,4-trimethyl pentane, which may be commercially obtained from Exxon-mobile under the trade name of Isopar C.

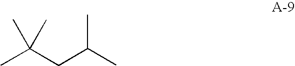

In various embodiments, the weight ratio between the polyalkylene such as polyethylene with M_{w} and the C₅₋₁₆ alkane may generally range from about 1:2 to about 1:8, typically range from about 1:3 to about 1:5. In a specific embodiment, the weight ratio between the polyalkylene such as polyethylene with M_{w} and the C₅₋₁₆ alkane is in the neighborhood of 1:4.

In typical embodiments, the separation method of this disclosure is scaleable. For example, in a single operation, at least 30 kg, typically at least 40 kg, more typically at least 50 kg of polyalkylene such as polyethylene with M_{w} (e.g. Polywax 2000) may be subject to the method.

In various embodiments, the steps (ii), (iii) and (iv) of the method may be conducted at an elevated temperature such as above room temperature, for example, from about 45° C. to about 125° C., more typically from about 65° C. to about 105° C. such as 85° C. In exemplary embodiments, the method of the present disclosure may be commonly called hot solvent extraction. In a specific embodiment, the method is a hot solvent extraction of virgin Polywax 2000 (PW2000) by Isopar C at 85° C.

If desired, commonly-known extraction techniques may be used in the method of the disclosure. For example, the method may be conducted with the aid of filter such as vacuum filter, dryer, or combination thereof such as Cogeim filter-dryer at XRCC pilot-plant; the method may also be conducted with stirring such as 30 RPM; the method may use a sufficiently long operation hour to obtain optimal separation result such as 1-6 hours, for example 3 hours; for a given sample, the method may be repeated as many times as desired, for example, 2-6 times such as 4 times. 4=12 hours; and the raw wax material and the purified wax material may be analyzed by DSC and High Temperature GPC (HTGPC).

Beneficially, the method according to this disclosure is easy to operate, highly reproducible. In exemplary embodiments, the method not only can solve the high temperature Gyricon tolerance problem, but it also alleviates the batch-to-batch variability of Polywax from Baker-Petrolite. This batch-to-batch variability has a negative effect on final device performance. The root cause is the variability in the distribution of Mw of Polywax. After implementation of the present method, narrowing of the Mw distribution is observed, and this eliminates the wax variability. Also, raw wax material has usually a broader melting characteristic. After the purification process of the method, it is shown that the melting point becomes sharper, which can possibly enhance the toner fusing properties and also the jetting conditions in SIJ project.

The disclosure further provides a microencapsulated gyricon bead comprising the separated/purified polyalkylene wax such as the second portion polyethylene with M_{w2} made from the method as illustrated above. Generally, the microencapsulated gyricon bead includes a bichromal sphere formed of a first material and a second material. A third liquid material such as transparent oil surrounds the bichromal sphere and functions as a rotation medium for the bichromal sphere. The bichromal sphere and the surrounding third material may be disposed within a fourth solid material.

The first material and the second material divide the bichromal sphere into two hemispheres. The hemispheres, namely the first material and the second material, are both optically isotropic and electrically isotropic. In various exemplary embodiments, the first material and the second material are pigmented plastics, with different surface colors between each other.

In various embodiments, the base polymer for one or two hemispheres of the bichromal sphere may comprise the purified polyalkylene wax of this disclosure such as purified Polywax 1000 and/or Polywax 2000. For example, a lighter or white pigment may be dispersed into the white/lighter hemisphere. Titanium dioxide white pigment such as is DuPont R104 TiO₂ pigment may be used for this purpose. On the black/color hemisphere of the bichromal sphere, a variety of black pigments may be used, such as manganese ferrite and carbon black, e.g. Ferro 6331 manufactured by the Ferro Corporation. Of course, other suitable pigments can also be used such as modified carbon blacks, magnetites, ferrites, and color pigments.

The bichromal spheres are relatively small, for example from about 2 to about 200 microns in diameter, and typically from about 30 to about 120 microns in diameter. In media that are active in an electric field, the bichromal spheres have a net dipole due to different levels of charge on the two sides of the sphere. An image is formed by the application of an electric field to the bichromal spheres, which rotates the bichromal spheres to expose one color or the other to the viewing surface of the media. The spheres may also have a net charge, in which case they will translate in the electric field as well as rotate. When the electric field is reduced or eliminated, the spheres ideally do not rotate further; hence, both colors of the image remain intact.

In some embodiments, crystalline materials are ideal for the production of high quality bichromal spheres. This is possibly due to the crystalline material's ability to transition rapidly from a low viscosity liquid to a solid as they cool by moving through the air. Unpurified polyalkylene has little or no crystalline properties. This is due to the relatively large size range of the molecules, but purified polyalkylene typically has stronger crystalline properties. By “crystalline”, it is referred to materials that remain solid as the temperature is increased. Specifically, when the melting point of the material is reached, a crystalline material will melt, sometimes abruptly, and become a low viscosity liquid. This is a desired feature of the crystalline material. For example, this property preserves the hemispherical bichromal quality of the beads after they are formed by the break-up of the Taylor instability jets formed on the edge of the spinning disk during manufacture.

In some embodiments, the purified polyalkylene wax such as Polywax 2000 of the disclosure is more desired if it has a linear structure and/or has a lower polydispersity such as PDI₁ and PDI₂, which aids in the material having a high crystalline property. Also desired are crystalline materials having a relatively low melting point of from about 50 to about 180° C., and more specifically from about 80 to about 130° C. Further, it is desirable that the crystalline material have a carbon content of from about 18 to about 1,000, and more specifically from about 50 to about 200 carbon atoms.

The fabrication of certain bichromal spheres is known, for example, as set forth in U.S. Pat. No. 4,143,103 patent, wherein the sphere is comprised of black polyethylene with a light reflective material, for example, indium, sputtered on one hemisphere. Also in U.S. Pat. No. 4,438,160, a rotary ball is prepared by coating white glass balls of about 50 microns in diameter, with an inorganic coloring layer such as co-deposited MgF₂ and chromium by evaporation. In a similar process, there is disclosed in an article entitled “The Gyricon—A twisting Ball Display”, published in the proceedings of the S.I.D., Vol. 18/3 and 4 (1977), a method for fabricating bichromal balls by first heavily loading chromatic glass balls with a white pigment such as titanium oxide, followed by coating from one direction in a vacuum evaporation chamber with a dense layer of nonconductive black material which coats only one hemisphere.

Also in U.S. Pat. No. 4,810,431 by Leidner, there is disclosed a process for generating spherical particles by (a) coextruding a fiber of a semi-circular layer of a polyethylene pigmented white and a black layer of polyethylene containing magnetite, (b) chopping the resultant fiber into fine particles ranging from 10 microns to about 10 millimeters, (c) mixing the particles with clay or anti-agglomeration materials, and (d) heating the mixture with a liquid at about 120° C. to spherodize the particles, followed by cooling to allow for solidification.

In another method, the bichromal beads used in the fabrication of display media such as Gyricon electric paper are formed by wetting the top and bottom surfaces of a spinning disk with two different pigmented molten solids. These streams combine at the edge of the disk and, driven by a Taylor instability, they form a series of jets emanating from the edge of the disk. In particular, a 3 inch diameter disk will have about 300 such jets. Each jet is seen with high speed video to be comprised of two very distinct parts corresponding to the two pigmented liquids used, with no apparent mixing within the jet. The jets subsequently break up into spheres by the Rayleigh instability. Again, with high speed video, it can be seen that close to the jet break-up points, these spheres are very high quality, hemispherical bichromal spheres.

The third material may be any dielectric liquid, such as the Isopars by the Exxon Corporation, and 1 or 2 centistoke silicone 200 liquid by the Dow Corning Corporation. The fourth material/skin may be any highly transparent and physically tough polymer with a temperature/viscosity profile that will allow it to house the bichromal sphere. Once again, the purified polyalkylene wax of this disclosure such as purified Polywax 1000 and/or Polywax 2000 may be used in the fourth material/skin.

A gyricon display may be prepared from the microencapsulated gyricon beads as illustrated above. Sometimes, gyricon displays are also known as electric paper, display media, or twisted ball panel display devices, and are described, for example, in U.S. Pat. Nos. 4,126,854; 4,143,103; 4,261,653; 4,438,160; 5,389,945. In an exemplary gyricon display, the microencapsulated gyricon beads are sandwiched between two indium tin oxide coated substrates, such as glass or MYLAR®.

A typical process for forming the bichromal balls described herein is as follows. After purification, the purified polyalkylene wax is mixed with a first pigment to produce a first wax material. The purified polyalkylene wax is mixed with a second pigment to produce a second wax material. These mixing operations can be performed to produce many different wax materials, typically having different colors or other different properties as compared to the other materials.

Next, the wax materials prepared are then heated to a temperature greater than the highest melting temperature of the wax materials. The heating operations can be performed separately upon each of the wax materials or collectively. Upon the wax materials being heated to a suitable temperature such that the wax material flows, the materials are then deposited onto a spinning disk to produce bichromal balls adapted for use in high temperature applications. The spinning disk production method is described in one or more of the patents referenced herein.

The polymer or wax materials can be colored through the addition of pigments, dyes, light reflective or light blocking particles, etc., as it is commonly known in the art. In this regard, a “pigment” is defined herein to include any substance, usually in the form of a dry powder, which imparts color to another substance or mixture. Most pigments are insoluble in organic solvents and water; exceptions are the natural organic pigments, such as chlorophyll, which are generally organosoluble. To qualify as a pigment, a material must have positive colorant value. This definition excludes whiting, barytes, clays, and talc.

Pigments may be classified as follows:
I. Inorganic
   (a) metallic oxides (iron, titanium, zinc, cobalt, chromium).
   (b) metal powder suspensions (gold, aluminum).
   (c) earth colors (siennas, ochers, umbers).
   (d) lead chromates.
   (e) carbon black.
II. Organic
   (a) animal (rhodopsin, melanin).
   (b) vegetable (chlorophyll, xantrophyll, indigo, flavone, carotene).

Some pigments (zinc oxide, carbon black) are also reinforcing agents, but the two terms are not synonymous; in the parlance of the paint and rubber industries these distinctions are not always observed.

“Dyes” include natural and synthetic dyes. A natural dye is an organic colorant obtained from an animal or plant source. Among the best-known are madder, cochineal, logwood, and indigo. The distinction between natural dyes and natural pigments is often arbitrary.

A synthetic dye is an organic colorant derived from coal-tar- and petroleum-based intermediates and applied by a variety of methods to impart bright, permanent colors to textile fibers. Some dyes, call “fugitive,” are unstable to sunlight, heat, and acids or bases; others, called “fast,” are not. Direct (or substantive) dyes can be used effectively without “assistants”; indirect dyes require either chemical reduction (vat type) or a third substance (mordant), usually a metal salt or tannic acid, to bind the dye to the fiber.

A “colorant” as used herein is any substance that imparts color to another material or mixture. Colorants are either dyes or pigments, and may either be (1) naturally present in a material, (2) admixed with it mechanically, or (3) applied to it in a solution.

There may be no generally accepted distinction between dyes and pigments. Some have proposed one on the basis of solubility, or of physical form and method of application. Most pigments, so called, are insoluble, inorganic powders, the coloring effect being a result of their dispersion in a solid or liquid medium. Most dyes, on the other hand, are soluble synthetic organic products which are chemically bound to and actually become part of the applied material. Organic dyes are usually brighter and more varied than pigments, but tend to be less stable to heat, sunlight, and chemical effects. The term colorant applies to black and white as well as to actual colors.

Examples of such colorants (i.e., pigments, dyes, etc.) and their commercial sources include, but are not limited to, magenta pigments such as 2,9-dimethyl-substituted quinacridone and anthraquinone dye, identified in the color index as C1 60710, C1 Dispersed Red 15, a diazo dye identified in the color index as C1 26050, C1 Solvent Red 19, and the like; cyan pigments including copper tetra-4-(octadecylsulfonamido) phthalocyanine, copper phthalocyanine pigment, listed in the color index as C1 74160, Pigment Blue, and Anthradanthrene Blue, identified in the color index as C1 69810, Special Blue X-2137, and the like; yellow pigments including diarylide yellow 3,3-dichlorobenzidine acetoacetanilides, a monoazo pigment identified in the color index as C1 12700, C1 Solvent Yellow 16, a nitrophenyl amine sulfonamide identified in the color index as Foron Yellow SE/GLN, C1 Dispersed Yellow 33, 2,5-dimethoxy acetoacetanilide, Permanent Yellow FGL, and the like. Other suitable colorants include Normandy Magenta RD-2400 (Paul Uhlich), Paliogen Violet 5100 (BASF), Paliogen Violet 5890 (BASF), Permanent Violet VT2645 (Paul Uhlich), Heliogen Green L8730 (BASF), Argyle Green XP-111-S (Paul Uhlich), Brilliant Green Toner GR 0991 (Paul Uhlich), Heliogen Blue L6900, L7020 (BASF), Heliogen Blue D6840, D7080 (BASF), Sudan Blue OS (BASF), PV Fast Blue B2G0 (American Hoechst), Irgalite Blue BCA (Ciba-Geigy), Paliogen Blue 6470 (BASF), Sudan III (Matheson, Coleman, Bell), Sudan II (Matheson, Coleman, Bell), Sudan IV (Matheson, Coleman, Bell), Sudan Orange G (Aldrich, Sudan Orange 220 (BASF), Paliogen Orange 3040 (BASF), Ortho Orange OR 2673 (Paul Uhlich), Paliogen Yellow 152, 1560 (BASF), Lithol Fast Yellow 0991K (BASF), Paliotol Yellow 1840 (BASF), Novoperm Yellow FG1 (Hoechst), Permanent Yellow YE 0305 (Paul Uhlich), Lumogen Yellow D0790 (BASF), Suco-Gelb L1250 (BASF), Suco-Yellow D1355 (BASF), Hostaperm Pink E (American Hoechst), Fanal Pink D4830 (BASF), Cinquasia Magenta (DuPont), Lithol Scarlet D3700 (BASF), Tolidine Red (Aldrich), Scarlet for Thermoplast NSD PS PA (Ugine Kuhlmann of Canada), E.D. Toluidine Red (Aldrich), Lithol Rubine Toner (Paul Uhlich), Lithol Scarlet 4440 (BASF), Bon Red C (Dominion Color Co.), Royal Brilliant Red RD-8192 (Paul Uhlich), Oracet Pink RF (Ciba-Geigy), Paliogen Red 3871 K (BASF), Paliogen Red 3340 (BASF), and Lithol Fast Scarlet L4300 (BASF). Examples of black pigments include carbon black products from Cabot corporation, such as Black Pearls 2000, Black Pearls 1400, Black Pearls 1300, Black Pearls 1100, Black Pearls 1000, Black Pearls 900, Black Pearls 880, Black Pearls 800, Black Pearls 700, Black Pearls 570, Black Pearls 520, Black Pearls 490, Black Pearls 480, Black Pearls 470, Black Pearls 460, Black Pearls 450, Black Pearls 430, Black Pearls 420, Black Pearls 410, Black Pearls 280, Black Pearls 170, Black Pearls 160, Black Pearls 130, Black Pearls 120, Black Pearls L; Vulcan XC72, Vulcan PA90, Vulcan 9A32, Regal 660, Regal 400, Regal 330, Regal 350, Regal 250, Regal 991, Elftex pellets 115, Mogul L.

Carbon black products from Degussa-Hüls such as FW1, Nipex 150, Printex 95, SB4, SB5, SB100, SB250, SB350, SB550; Carbon black products from Columbian such as Raven 5750; Carbon black products from Mitsubishi Chemical such as #25, #25B, #44, and MA-100-S can also be utilized.

Other black pigments that may also be used include Ferro™ 6330, a manganese ferrite pigment available from Ferro Corporation, and Paliotol Black 0080 (Aniline Black) available from BASF.

Moreover, one or more processing aid, such as surface active agents and dispersants aids like Aerosol™ OT-100 (from American Cynamid Co. of Wayne, N.J.) and aluminum octoate (Witco). Dispersant aids such as X-5175 (from Baker-Petrolite Corporation), Unithox™ 480 (from Baker-Petrolite Corp.), Polyox™ N80 (Dow), and Ceramer™ 5750 (Baker-Petrolite Corp.) can also be added to the waxy base material.

Once the high temperature bichromal balls are produced by the process set forth above, they may be encapsulated for use in high temperature display applications. Generally, the encapsulation process involves providing a silicone oil which as previously noted can be polydimethylsiloxane. A shell material as described in the art is also provided. The high temperature bichromal balls, i.e. those utilizing the purified polyalkylene wax, are then encapsulated. The bichromal balls are dispersed in the silicone oil within a shell of the shell material.

The present disclosure is also directed to a phase change ink, alternatively known as solid ink or hot melt ink. In various embodiments, the phase change ink contains a colorant and a carrier comprising the purified polyalkylene wax such as purified Polywax 1000 and/or Polywax 2000, as described above.

Any desired or effective colorant may be employed in the phase change inks of the present disclosure, including dyes, pigments, mixtures thereof, and the like, provided that the colorant can be dissolved or dispersed in the phase change ink carrier.

In various embodiments, the carrier comprising the purified polyalkylene wax of this disclosure may be combined with one or more of compatible subtractive primary colorants. The subtractive primary colored phase change inks may comprise four component dyes, namely, cyan, magenta, yellow and black, although the inks are not limited to these four colors. These subtractive primary colored inks can be formed by using a single dye or a mixture of dyes. For example, magenta can be obtained by using a mixture of Solvent Red Dyes or a composite black can be obtained by mixing several dyes. U.S. Pat. No. 4,889,560, U.S. Pat. No. 4,889,761, and U.S. Pat. No. 5,372,852, the disclosures of each of which are totally incorporated herein by reference, teach that the subtractive primary colorants employed can comprise dyes from the classes of Color Index (C.I.) Solvent Dyes, Disperse Dyes, modified Acid and Direct Dyes, and Basic Dyes. The colorants can also include pigments, as disclosed in, for example, U.S. Pat. No. 5,221,335, the disclosure of which is totally incorporated herein by reference. U.S. Pat. No. 5,621,022, the disclosure of which is totally incorporated herein by reference, discloses the use of a specific class of polymeric dyes in phase change ink compositions.

In various embodiments, conventional phase change ink colorant materials may be used, such as Color Index (C.I.) Solvent Dyes, Disperse Dyes, modified Acid and Direct Dyes, Basic Dyes, Sulphur Dyes, Vat Dyes, and the like. Examples of suitable dyes include Neozopon Red 492 (BASF); Orasol Red G (Ciba-Geigy); Direct Brilliant Pink B (Crompton & Knowles); Aizen Spilon Red C-BH (Hodogaya Chemical); Kayanol Red 3BL (Nippon Kayaku); Levanol Brilliant Red 3BW (Mobay Chemical); Levaderm Lemon Yellow (Mobay Chemical); Spirit Fast Yellow 3G; Aizen Spilon Yellow C-GNH (Hodogaya Chemical); Sirius Supra Yellow GD 167; Cartasol Brilliant Yellow 4GF (Sandoz): Pergasol Yellow CGP (Ciba-Geigy); Orasol Black RLP (Ciba-Geigy); Savinyl Black RLS (Sandoz); Dermacarbon 2GT (Sandoz); Pyrazol Black BG (ICI); Morfast Black Conc. A (Morton-Thiokol): Dioazol Black RN Quad (ICI); Orasol Blue GN (Ciba-Geigy); Savinyl Blue GLS (Sandoz); Luxol Blue MBSN (Morton-Thiokol); Sevron Blue 5GMF (ICI); Basacid Blue 750 (BASF), Neozapon Black X51 [C.I. Solvent Black, C.I. 12195] (BASF), Sudan Blue 670 [C.I. 61554] (BASF), Sudan Yellow 146 [C.I. 12700] (BASF), Sudan Red 462 [C.I. 26050] (BASF), Intratherm Yellow 346 from Crompton and Knowles, C.I. Disperse Yellow 238, Neptune Red Base NB543 (BASF, C.I. Solvent Red 49), Neopen Blue FF-4012 from BASF, Lampronol Black BR from ICI (C.I. Solvent Black 35), Morton Morplas Magenta 36 (C.I. Solvent Red 172), metal phthalocyanine colorants such as those disclosed in U.S. Pat. No. 6,221,137, the disclosure of which is totally incorporated herein by reference, and the like. Polymeric dyes can also be used, such as those disclosed in, for example, U.S. Pat. Nos. 5,621,022 and 5,231,135, the disclosures of each of which are totally incorporated herein by reference, and commercially available from, for example, Milliken & Company as Milliken Ink Yellow 869, Milliken Ink Blue 92, Milliken Ink Red 357, Milliken Ink Yellow 1800, Milliken Ink Black 8915-67, uncut Reactant Orange X-38, uncut Reactant Blue X-17, and uncut Reactant Violet X-80.

Pigments are also suitable colorants for the phase change inks of the present invention. Examples of suitable pigments include Violet Toner VT-8015 (Paul Uhlich); Paliogen Violet 5100 (BASF); Paliogen Violet 5890 (BASF); Permanent Violet VT 2645 (Paul Uhlich); Heliogen Green L8730 (BASF); Argyle Green XP-111-S (Paul Uhlich); Brilliant Green Toner GR 0991 (Paul Uhlich); Lithol Scarlet D3700 (BASF); Toluidine Red (Aldrich); Scarlet for Thermoplast NSD PS PA (Ugine Kuhlmann of Canada): E.D. Toluidine Red (Aldrich): Lithol Rubine Toner (Paul Uhlich): Lithol Scarlet 4440 (BASF); Bon Red C (Dominion Color Company); Royal Brilliant Red RD8192 (Paul Uhlich); Oracet Pink RF (Ciba-Geigy); Paliogen Red 3871 K (BASF); Paliogen Red 3340 (BASF); Lithol Fast Scarlet L4300 (BASF); Heliogen Blue L6900, L7020 (BASF); Heliogen Blue K6902, K6910 (BASF); Heliogen Blue D6840, D7080 (BASF); Sudan Blue OS (BASF); Neopen Blue FF4012 (BASF); PV Fast Blue B2GO1 (American Hoechst); Irgalite Blue BCA (Ciba-Geigy): Paliogen Blue 6470 (BASF): Sudan III (Red Orange) (Matheson, Colemen Bell); Sudan II (Orange) (Matheson, Colemen Bell); Sudan Orange G (Aldrich). Sudan Orange 220 (BASF); Paliogen Orange 3040 (BASF); Ortho Orange OR 2673 (Paul Uhlich); Paliogen Yellow 152, 1560 (BASF); Lithol Fast Yellow 0991 K (BASF); Paliotol Yellow 1840 (BASF); Novoperm Yellow FGL (Hoechst); Permanent Yellow YE 0305 (Paul Uhlich); Lumogen Yellow D0790 (BASF); Suco-Yellow L1250 (BASF); Suco-Yellow D1355 (BASF); Suco Fast Yellow D1355, D1351 (BASF); Hostaperm Pink E (American Hoechst): Fanal Pink D4830 (BASF): Cinquasia Magenta (DuPont); Paliogen Black L0084 (BASF); Pigment Black K801 (BASF); and carbon blacks such as REGAL 3300 (Cabot), Carbon Black 5250, Carbon Black 5750 (Columbia Chemical), and the like.

Also suitable as colorants are the isocyanate-derived colored resins disclosed in U.S. Pat. No. 5,780,528, the disclosure of which is totally incorporated herein by reference.

Also suitable are the colorants disclosed in U.S. application Ser. No. 10/072,241, filed Feb. 8, 2002, entitled “Phthalocyanine Compositions”; U.S. application Ser. No. 10/072,210, Feb. 8, 2002, entitled “Ink Compositions Containing Phthalocyanines”; U.S. application Ser. No. 10/072,237, filed Feb. 8, 2002, entitled “Methods For Preparing Phthalocyanine Compositions”; U.S. application Ser. No. 10/185,261, filed Jun. 27, 2002, entitled “Processes for Preparing Dianthranilate Compounds and Diazopyridone Colorants”; U.S. application Ser. No. 10/185,994, filed Jun. 27, 2002, entitled “Dimeric Azo Pyridone Colorants”; U.S. application Ser. No. 10/184,269, filed Jun. 27, 2002, entitled “Phase Change Inks Containing Dimeric Azo Pyridone Colorants”; U.S. application Ser. No. 10/185,264, filed Jun. 27, 2002, entitled “Phase Change Inks Containing Azo Pyridone Colorants”; U.S. application Ser. No. 10/186,024, filed Jun. 27, 2002, entitled “Azo Pyridone Colorants”; U.S. application Ser. No. 10/185,597, filed Jun. 27, 2002, entitled “Process for Preparing Substituted Pyridone Compounds”; U.S. application Ser. No. 10/185,828, filed Jun. 27, 2002, entitled “Method for Making Dimeric Azo Pyridone Colorants”; U.S. application Ser. No. 10/186,023, filed Jun. 27, 2002, entitled “Dimeric Azo Pyridone Colorants”; and U.S. application Ser. No. 10/184,266, filed Jun. 27, 2002, entitled “Phase Change Inks Containing Dimeric Azo Pyridone Colorants”, the disclosures of each of which are totally incorporated herein by reference.

Other ink colors besides the subtractive primary colors can be desirable for applications such as postal marking or industrial marking and labeling using phase change printing, and the present invention is applicable to these needs. Further, infrared (IR) or ultraviolet (UV) absorbing dyes can also be incorporated into the inks of the present invention for use in applications such as “invisible” coding or marking of products. Examples of such infrared and ultraviolet absorbing dyes are disclosed in, for example, U.S. Pat. Nos. 5,378,574, 5,146,087, 5,145,518, 5,543,177, 5,225,900, 5,301,044, 5,286,286, 5,275,647, 5,208,630, 5,202,265, 5,271,764, 5,256,193, 5,385,803, and 5,554,480, the disclosures of each of which are totally incorporated herein by reference.

The colorant is present in the phase change ink of the present invention in any desired or effective amount to obtain the desired color or hue, in one embodiment at least about 0.1 percent by weight of the ink, in another embodiment at least about 0.5 percent by weight of the ink, and in yet another embodiment at least about 2 percent by weight of the ink, and in one embodiment no more than about 15 percent by weight of the ink, in another embodiment no more than about 10 percent by weight of the ink, in yet another embodiment no more than about 8 percent by weight of the ink, and in still another embodiment no more than about 6 percent by weight of the ink, although the amount can be outside of these ranges.

The carrier of the phase change ink according to this disclosure is typically a composition comprising the purified polyalkylene wax such as purified Polywax 1000 and/or Polywax 2000, as described above. The carrier is designed for use in either a direct printing mode or an indirect or offset printing transfer system.

In the direct printing mode, the phase change carrier composition in one embodiment contains one or more materials that enable the phase change ink (1) to be applied in a thin film of uniform thickness on the final recording substrate (such as paper, transparency material, and the like) when cooled to ambient temperature after printing directly to the recording substrate, (2) to be ductile while retaining sufficient flexibility so that the applied image on the substrate will not fracture upon bending, and (3) to possess a high degree of lightness, chroma, transparency, and thermal stability.

In an offset printing transfer or indirect printing mode, the phase change carrier composition in one embodiment exhibits not only the characteristics desirable for direct printing mode inks, but also certain fluidic and mechanical properties desirable for use in such a system, as described in, for example, U.S. Pat. No. 5,389,958 the disclosure of which is totally incorporated herein by reference.

Optimally, one or more of any other desired or effective carrier material may be combined with the purified polyalkylene wax such as purified Polywax 1000 and/or Polywax 2000, as described above, in formulating the phase change ink of the disclosure.

Examples of other suitable carrier materials include fatty amides, such as monoamides, tetra-amides, mixtures thereof, and the like. Specific examples of suitable fatty amide ink carrier materials include stearyl stearamide, a dimer acid based tetra-amide that is the reaction product of dimer acid, ethylene diamine, and stearic acid, a dimer acid based tetra-amide that is the reaction product of dimer acid, ethylene diamine, and a carboxylic acid having at least about 36 carbon atoms, and the like, as well as mixtures thereof. When the fatty amide ink carrier is a dimer acid based tetra-amide that is the reaction product of dimer acid, ethylene diamine, and a carboxylic acid having at least about 36 carbon atoms, the carboxylic acid is of the general formula as shown below. 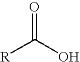
wherein R is an alkyl group, including linear, branched, saturated, unsaturated, and cyclic alkyl groups, said alkyl group in one embodiment having at least about 36 carbon atoms, in another embodiment having at least about 40 carbon atoms, said alkyl group in one embodiment having no more than about 200 carbon atoms, in another embodiment having no more than about 150 carbon atoms, and in yet another embodiment having no more than about 100 carbon atoms, although the number of carbon atoms can be outside of these ranges. Carboxylic acids of this formula are commercially available from, for example, Baker Petrolite, Tulsa, Okla., and can also be prepared as described in Example 1 of U.S. Pat. No. 6,174,937, the disclosure of which is totally incorporated herein by reference. Further information on fatty amide carrier materials is disclosed in, for example, U.S. Pat. No. 4,889,560, U.S. Pat. No. 4,889,761, U.S. Pat. No. 5,194,638, U.S. Pat. No. 4,830,671, U.S. Pat. No. 6,174,937, U.S. Pat. No. 5,372,852, U.S. Pat. No. 5,597,856, U.S. Pat. No. 6,174,937, and British Patent GB 2 238 792, the disclosures of each of which are totally incorporated herein by reference.

Yet other suitable carrier materials are isocyanate-derived resins and waxes, such as urethane isocyanate-derived materials, urea isocyanate-derived materials, urethane/urea isocyanate-derived materials, mixtures thereof, and the like. Further information on isocyanate-derived carrier materials is disclosed in, for example, U.S. Pat. No. 5,750,604, U.S. Pat. No. 5,780,528, U.S. Pat. No. 5,782,966, U.S. Pat. No. 5,783,658, U.S. Pat. No. 5,827,918, U.S. Pat. No. 5,830,942, U.S. Pat. No. 5,919,839, U.S. Pat. No. 6,255,432, U.S. Pat. No. 6,309,453, British Patent GB 2 294 939, British Patent GB 2 305 928, British Patent GB 2 305 670, British Patent GB 2 290 793, PCT Publication WO 94/14902, PCT Publication WO 97/12003, PCT Publication WO 97/13816, PCT Publication WO 96/14364, PCT. Publication WO 97/33943, and PCT Publication WO 95/04760, the disclosures of each of which ore totally incorporated herein by reference.

Additional suitable carrier materials include ester waxes, amide waxes, fatty acids, fatty alcohols, fatty amides and other waxy materials, sulfonamide materials, resinous materials made from different natural sources (such as, for example, tall oil rosins and rosin esters), and many synthetic resins, oligomers, polymers and copolymers, such as ethylene/vinyl acetate copolymers, ethylene/acrylic acid copolymers, ethylene/vinyl acetate/acrylic acid copolymers, copolymers of acrylic acid with polyamides, and the like, ionomers, and the like, as well as mixtures thereof.

The carrier composition is present in the phase change ink of the present invention in any desired or effective amount, in one embodiment of at least about 0.1 percent by weight of the ink, in another embodiment of at least about 50 percent by weight of the ink, and in yet another embodiment of at least about 90 percent by weight of the ink, and in one embodiment of no more than about 99 percent by weight of the ink, in another embodiment of no more than about 98 percent by weight of the ink, and in yet another embodiment of no more than about 95 percent by weight of the ink, although the amount can be outside of these ranges.

The phase change inks of the present invention can also optionally contain an antioxidant. The optional antioxidants protect the images from oxidation and also protect the ink components from oxidation during the heating portion of the ink preparation process. Specific examples of suitable antioxidants include NAUGUARD® 524, NAUGUARD® 76, and NAUGUARD® 512 (commercially available from Uniroyal Chemical Company, Oxford, Conn.), IRGANOX®0 1010 (commercially available from Ciba Geigy), and the like. When present, the optional antioxidant is present in the ink in any desired or effective amount, in one embodiment of at least about 0.01 percent by weight of the ink, in another embodiment of at least about 0.1 percent by weight of the ink, and in yet another embodiment of at least about 1 percent by weight of the ink, and in one embodiment of no more than about 20 percent by weight of the ink, in another embodiment of no more than about 5 percent by weight of the ink, and in yet another embodiment of no more than about 3 percent by weight of the ink, although the amount can be outside of these ranges.

In one specific embodiment, the phase change ink carrier comprises (a) the purified polyalkylene wax such as polyethylene wax, e.g. purified Polywax 1000 and/or Polywax 2000, as described above, present in the ink in an amount in one embodiment of at least about 25 percent by weight of the ink, in another embodiment of at least about 30 percent by weight of the ink, and in yet another embodiment of at least about 37 percent by weight of the ink, and in one embodiment of no more than about 60 percent by weight of the ink, in another embodiment of no more than about 53 percent by weight of the ink, and in yet another embodiment of no more than about 48 percent by weight of the ink, although the amount can be outside of these ranges; (b) a stearyl stearamide wax, present in the ink in an amount in one embodiment of at least about 8 percent by weight of the ink, in another embodiment of at least about 10 percent by weight of the ink, and in yet another embodiment of at least about 12 percent by weight of the ink, and in one embodiment of no more than about 32 percent by weight of the ink, in another embodiment of no more than about 28 percent by weight of the ink, and in yet another embodiment of no more than about 25 percent by weight of the ink, although the amount can be outside of these ranges; (c) a dimer acid based tetra-amide that is the reaction product of dimer acid, ethylene diamine, and a long chain hydrocarbon having greater than thirty six carbon atoms and having a terminal carboxylic acid group, present in the ink in an amount in one embodiment of at least about 10 percent by weight of the ink in another embodiment of at least about 13 percent by weight of the ink, and in yet another embodiment of at least about 16 percent by weight of the ink, and in one embodiment of no more than about 32 percent by weight of the ink, in another embodiment of no more than about 27 percent by weight of the ink, and in yet another embodiment of no more than about 22 percent by weight of the ink, although the amount can be outside of these ranges (d) a urethane resin derived from the reaction of two equivalents of hydroabietyl alcohol and one equivalent of isophorone diisocyanate, present in the, ink in an amount in one embodiment of at least about 6 percent by weight of the ink, in another embodiment of at least about 8 percent by weight of the ink, and in yet another embodiment of at least about 10 percent by weight of the ink, and in one embodiment of no more than about 16 percent by weight of the ink, in another embodiment of no more than about 14 percent by weight of the ink, and in yet another embodiment of no more than about 12 percent by weight of the ink, although the amount can be outside of these ranges; (e) a urethane resin that is the adduct of three equivalents of stearyl isocyanate and a glycerol-based propoxylate alcohol, present in the ink in an amount in one embodiment of at least about 2 percent by weight of the ink, in another embodiment of at least about 3 percent by weight of the ink, and in yet another embodiment of at least about 4.5 percent by weight of the ink, and in one embodiment of no more than about 13 percent by weight of the ink, in another embodiment of no more than about 10 percent by weight of the ink, and in yet another embodiment of no more than about 7.5 percent by weight of the ink, although the amount can be outside of these ranges; and (f) an antioxidant, present in the ink in an amount in one embodiment of at least about 0.01 percent by weight of the ink, in another embodiment of at least about 0.05 percent by weight of the ink, and in yet another embodiment of at least about 0.1 percent by weight of the ink, and in one embodiment of no more than about 1 percent by weight of the ink, in another embodiment of no more than about 0.5 percent by weight of the ink, and in yet another embodiment of no more than about 0.3 percent by weight of the ink, although the amount can be outside of these ranges.

The phase change inks of the present invention can also optionally contain a viscosity modifier. Examples of suitable viscosity modifiers include aliphatic ketones, such as stearone, and the like. When present, the optional viscosity modifier is present in the ink in any desired or effective amount, in one embodiment of at least about 0.1 percent by weight of the ink; in another embodiment of at least about 1 percent by weight of the ink, and in yet another embodiment of at least about 10 percent by weight of the ink, and in one embodiment of no more than about 99 percent by, weight of the ink, in another embodiment of no more than about 30 percent by weight of the ink, and in yet another embodiment of no more than about 15 percent by weight of the ink, although the amount can be outside of these ranges.

Other optional additives to the phase change inks include clarifiers, such as UNION CAMP® X37-523-235 (commercially available from Union Camp), in an amount in one embodiment of at least about 0.01 percent by weight of the ink, in another embodiment of at least about 0.1 percent by weight of the ink, and in yet another embodiment of at least about 5 percent by weight of the ink, and in one embodiment of no more than about 98 percent by weight of the ink, in another embodiment of no more than about 50 percent by weight of the ink, and in yet another embodiment of no more than about 10 percent by weight of the ink, although the amount can be outside of these ranges; tackifiers, such as FORAL® 85, a glycerol ester of hydrogenated abietic (rosin) acid (commercially available from Hercules), FORAL® 105, a pentaerythritol ester of hydroabietic (rosin) acid (commercially available from Hercules), CELLOLYN® 21, a hydroabietic (rosin) alcohol ester of phthalic acid (commercially available from Hercules), ARAKAWA KE-311 Resin, a triglyceride of hydrogenated abietic (rosin) acid (commercially available from Arakawa Chemical Industries, Ltd.), synthetic polyterpene resins such as NEVTAC® 2300, NEVTAC® 100, and NEVTAC® 80 (commercially available from Neville Chemical Company), WINGTACK® 86, a modified, synthetic polyterpene resin (commercially available from, Goodyear), and the like, in an amount in one embodiment of at least about 0.1 percent by weight of the ink, in another embodiment of at least about 5 percent by weight of the ink, and in yet another embodiment of at least about 10 percent by weight of the ink, and in one embodiment of no more than about 98 percent by weight of the ink, in another embodiment of no more than about 75 percent by weight of the ink, and in yet another embodiment of no more than about 50 percent by weight of the ink, although the amount can be outside of these range; adhesives, such as VERSAMID® 757, 759, or 744 (commercially available from Henkel), in an amount in one embodiment of at least about 0.1 percent by weight of the ink, in another embodiment of at least about 1 percent by weight of the ink, and in yet another embodiment of at least about 5 percent by weight of the ink, and in one embodiment of no more than about 98 percent by weight of the ink, in another embodiment of no more than about 50 percent by weight of the ink, and in yet another embodiment of no more than about 10 percent by weight of the ink, although the amount can be outside of these ranges; plasticizers, such as UNIPLEX® 250 (commercially available from Uniplex) the phthalate ester plasticizers commercially available from Monsanto under the trade name SANTICIZER®, such as dioctyl phthalate, diundecyl phthalate, alkylbenzyl phthalate (SANTICIZER® 278), triphenyl phosphate (commercially available from Monsanto), KP-140®, a tributoxyethyl phosphate (commercially available from FMC Corporation), MORFLEX® 150, a dicyclohexyl phthalate (commercially available from Morflex Chemical Company Inc.), trioctyl trimellitate (commercially available from Eastman Kodak Co.), and the like, in an amount in one embodiment of at least about 0.1 percent by weight of the ink, in another embodiment of at least about 1 percent by weight of the ink, and in yet another embodiment of at least about 2 percent by weight of the ink, and in one embodiment of no more than about 50 percent by weight of the ink, in another embodiment of no more than about 30 percent by weight of the ink, and in yet another embodiment of no more than about 10 percent by weight of the ink, although the amount can be outside of these ranges; and the like.

The phase change inks of the present invention in one embodiment have melting points of no lower than about 50° C., in another embodiment of no lower than about 70° C., and in yet another embodiment of no lower than about 80° C., and have melting points in one embodiment of no higher than about 160° C., in another embodiment of no higher than about 140° C., and in yet another embodiment of no higher than about 100° C., although the melting point can be outside of these ranges.

The phase change ink of the present invention generally have melt viscosities at the jetting temperature (in one embodiment no lower than about 75° C., in another embodiment no lower than about 100° C., and in yet another embodiment no lower than about 120° C., and in one embodiment no higher than about 180° C., and in another embodiment no higher than about 150° C., although the jetting temperature can be outside of these ranges) in one embodiment of no more than about 30 centipoise, in another embodiment of no more than about 20 centipoise, and in yet another embodiment of no more than about 15 centipoise, and in one embodiment of no less than about 2 centipoise, in another embodiment of no less than about 5 centipoise, and in yet another embodiment of no less than about 7 centipoise, although the melt viscosity can be outside of these ranges.

The phase change inks of the present invention can be prepared by any desired or suitable method. For example, the ink ingredients can be mixed together, followed by heating, to a temperature in one embodiment of at least about 100° C., and in one embodiment of no more than about 140° C., although the temperature can be outside of these ranges, and stirring until a homogeneous ink composition is obtained, followed by cooling the ink to ambient temperature (typically from about 20 to about 25° C.). The inks of the present invention are solid at ambient temperature. In a specific embodiment, during the formation process, the inks in their molten state are poured into molds and then allowed to cool and solidify to form ink sticks.

The phase change inks of the present invention can be employed in apparatus for direct printing ink jet processes and in indirect (offset) printing ink jet applications. Another embodiment is directed to a process which comprises incorporating an ink of the present invention into an ink jet printing apparatus, melting the ink, and causing droplets of the melted ink to be ejected in an imagewise pattern onto a recording substrate. A direct printing process is also disclosed in, for example, U.S. Pat. No. 5,195,430, the disclosure of which is totally incorporated herein by reference. Yet another embodiment of the present invention is directed to a process which comprises incorporating an ink of the present invention into an ink jet printing apparatus, melting the ink, causing droplets of the melted ink to be ejected in an imagewise pattern onto an intermediate transfer member, and transferring the ink in the imagewise pattern from the intermediate transfer member to a final recording substrate. In a specific embodiment, the intermediate transfer member is heated to a temperature above that of the final recording sheet and below that of the melted ink in the printing apparatus. An offset or indirect printing process is also disclosed in, for example, U.S. Pat. No. 5,389,958, the disclosure of which is totally incorporated herein by reference. In one specific embodiment, the printing apparatus employs a piezoelectric printing process wherein droplets of the ink are caused to be ejected in imagewise pattern by oscillations of piezoelectric vibrating elements. Inks of the present invention can also be employed in other hot melt printing processes, such as hot melt acoustic ink jet printing, hot melt thermal ink jet printing, hot melt continuous stream or deflection ink jet printing, and the like. Phase change inks of the present invention can also be used in printing processes other than hot melt ink jet printing processes.

Phase change ink printers conventionally receive ink in a solid form and convert the ink to a liquid form for jetting onto a receiving medium. The printer receives the solid ink either as pellets or as ink sticks in a feed channel. In a printer that receives solid ink sticks, the sticks are either gravity fed or spring loaded into a feed channel and pressed against a heater plate to melt the solid ink into its liquid form. U.S. Pat. No. 5,734,402 for a Solid Ink Feed System, issued Mar. 31, 1998 to Rousseau et al.; and U.S. Pat. No. 5,861,903 for an Ink Feed System, issued Jan. 19, 1999 to Crawford et al. describe exemplary systems for delivering solid ink sticks into a phase change ink printer.

Any suitable substrate or recording sheet can be employed, including plain papers such as XEROX® 4024 papers, XEROX® Image Series papers, Courtland 4024 DP paper, ruled notebook paper, bond paper, silica coated papers such as Sharp Company silica coated paper, JuJo paper, HAMMERMILL LASERPRINT™ paper, and the like, transparency materials, fabrics, textile products, plastics, polymeric films, inorganic substrates such as metals and wood, and the like.

The disclosure further provides an E/A toner, which is prepared from a toner formulation comprising a latex, a colorant dispersion, a coagulant, and a wax dispersion comprising the purified polyalkylene wax of this disclosure such as purified Polywax 1000 and/or Polywax 2000. Optionally, the toner formulation comprises silica, a charge enhancing additive or charge control additive, a surfactant, an emulsifier, a flow additive, and the mixture thereof.

The latex in the toner formulation may be prepared from any suitable monomers. Exemplary monomers include, but are not limited to, styrene, alkyl acrylate such as methyl acrylate, ethyl acrylate, butyl arylate, isobutyl acrylate, dodecyl acrylate, n-octyl acrylate, 2-chloroethyl acrylate; β-carboxy ethyl acrylate (β-CEA), phenyl acrylate, methyl alphachloroacrylate, methyl methacrylate, ethyl methacrylate, butyl methacrylate, butadiene, isoprene; methacrylonitrile, acrylonitrile; vinyl ethers such as vinyl methyl ether, vinyl isobutyl ether, vinyl ethyl ether and the like; vinyl esters such as vinyl acetate, vinyl propionate, vinyl benzoate, vinyl butyrate; vinyl ketones such as vinyl methyl ketone, vinyl hexyl ketone, methyl isopropenyl ketone and the like; vinylidene halides such as vinylidene chloride, vinylidene chlorofluoride and the like; N-vinyl indole, N-vinyl pyrrolidene and the like; methacrylate, acrylic acid, methacrylic acid, acrylamide, methacrylamide, vinylpyridine, vinylpyrrolidone, vinyl-N-methylpyridinium chloride, vinyl naphthalene, p-chlorostyrene, vinyl chloride, vinyl bromide, vinyl fluoride, ethylene, propylene, butylene, isobutylene, and the like, and the mixture thereof.

In typical embodiments, the latex in the toner formulation is a copolymer of two or more monomers. Illustrative examples of such latex copolymer include poly(styrene-n-butyl acrylate-β-CEA), poly(styrene-alkyl acrylate), poly(styrene-1,3-diene), poly(styrene-alkyl methacrylate), poly(alkyl methacrylate-alkyl acrylate), poly(alkyl methacrylate-aryl acrylate), poly(aryl methacrylate-alkyl acrylate), poly(alkyl methacrylate), poly(styrene-alkyl acrylate-acrylon itrile), poly(styrene-1,3-diene-acrylonitrile), poly(alkyl acrylate-acrylonitrile), poly(styrene-butadiene), poly(methylstyrene-butadiene), poly(methyl methacrylate-butadiene), poly(ethyl methacrylate-butadiene), poly(propyl methacrylate-butadiene), poly(butyl methacrylate-butadiene), poly(methyl acrylate-butadiene), poly(ethyl acrylate-butadiene), poly(propyl acrylate-butadiene), poly(butyl acrylate-butadiene), poly(styrene-isoprene), poly(methylstyrene-isoprene), poly(methyl methacrylate-isoprene), poly(ethyl methacrylate-isoprene), poly(propyl methacrylate-isoprene), poly(butyl methacrylate-isoprene), poly(methyl acrylate-isoprene), poly(ethyl acrylate-isoprene), poly(propyl acrylate-isoprene), poly(butyl acrylate-isoprene); poly(styrene-propyl acrylate), poly(styrene-butyl acrylate), poly(styrene-butadiene-acrylonitrile), poly(styrene-butyl acrylate-acrylononitrile), and the like.

Based on the total toner weight, the latex may generally be present in an amount from about 70% to about 90%, including from about 75% to about 90%, although it may be present in greater or lesser amounts.

The colorant in the toner formulation may be any colorant suitable for toner applications. Examples of suitable colorants include dyes and pigments, such as carbon black (for example, REGAL 330®), magnetites, phthalocyanines, HELIOGEN BLUE L6900, D6840, D7080, D7020, PYLAM OIL BLUE, PYLAM OIL YELLOW, and PIGMENT BLUE 1, all available from Paul Uhlich & Co., PIGMENT VIOLET 1, PIGMENT RED 48, LEMON CHROME YELLOW DCC 1026, E.D. TOLUIDINE RED, and BON RED C, all available from Dominion Color Co., NOVAPERM YELLOW FGL and HOSTAPERM PINK E, available from Hoechst, CINQUASIA MAGENTA, available from E.I. DuPont de Nemours & Company, 2,9-dimethyl-substituted quinacridone and anthraquinone dyes identified in the Color Index as C1 60710, C1 Dispersed Red 15, diazo dyes identified in the Color Index as C1 26050, C1 Solvent Red 19, copper tetra (octadecyl sulfonamido) phthalocyanine, x-copper phthalocyanine pigment listed in the Color Index as C1 74160, C1 Pigment Blue, Anthrathrene Blue, identified in the Color Index as C1 69810, Special Blue X-2137, diarylide yellow 3,3-dichlorobenzidene acetoacetanilides, a monoazo pigment identified in the Color Index as C1 12700, C1 Solvent Yellow 16, a nitrophenyl amine sulfonamide identified in the Color Index as Foron Yellow SE/GLN, C1 Dispersed Yellow 33 2,5-dimethoxy-4-sulfonanilide phenylazo-4′-chloro-2,5-dimethoxy acetoacetanilide, Permanent Yellow FGL, Pigment Yellow 74, B 15:3 cyan pigment dispersion, commercially available from Sun Chemicals, Magenta Red 81:3 pigment dispersion, commercially available from Sun Chemicals, Yellow 180 pigment dispersion, commercially available from Sun Chemicals, colored magnetites, such as mixtures of MAPICO BLACK® and cyan components, and the like, as well as mixtures thereof. Other commercial sources of pigments available as aqueous pigment dispersion from either Sun Chemical or Ciba include (but are not limited to) Pigment Yellow 17, Pigment Yellow 14, Pigment Yellow 93, Yellow Pigment PY74, Pigment Violet 23, Pigment Violet 1, Pigment Green 7, Pigment Orange 36, Pigment Orange 21, Pigment Orange 16, Pigment Red 185, Pigment Red 122, Pigment Red 81:3, Pigment Blue 15:3, and Pigment Blue 61, and other pigments that enable reproduction of the maximum Pantone color space. Mixtures of colorants can also be employed.

Based on the total toner weight, the colorant or colorant mixture may generally be present in an amount from about 0.5% to about 30%, including from about 1% to about 10%, although it may be present in greater or lesser amounts.

The coagulant in the toner formulation may be any coagulant suitable for toner applications. Examples of coagulants include polyaluminum halides such as polyaluminum chloride (PAC), or the corresponding bromide, fluoride, or iodide, polyaluminum silicates such as polyaluminum sulfo silicate (PASS), and water soluble metal salts including aluminum chloride, aluminum nitrite, aluminum sulfate, potassium aluminum sulfate, calcium acetate, calcium chloride, calcium nitrite, calcium oxylate, calcium sulfate, magnesium acetate, magnesium nitrate, magnesium sulfate, zinc acetate, zinc nitrate, zinc sulfate and the like.

A very typical coagulant is PAC which is commercially available, and can be prepared by the controlled hydrolysis of aluminum chloride with sodium hydroxide. Generally, the PAC can be prepared by the addition of two moles of a base to one mole of aluminum chloride. The species is soluble and stable when dissolved and stored under acidic conditions if the pH is less than 5. The species in solution is believed to be of the formula Al₁₃O₄(OH)₂₄ (H₂O)₁₂ with 7 positive electrical charges per unit.

Based on the total toner weight, the coagulant or coagulant mixture may generally be present in an amount from about 1% to about 10%, although it may be present in greater or lesser amounts.

The wax dispersion in the toner formulation comprises the purified polyalkylene wax of this disclosure such as purified Polywax 1000 and/or Polywax 2000. Optionally, other waxes suitable for toner applications may be combined with the purified polyalkylene wax of this disclosure. Various examples of other suitable waxes include, but are not limited to, Fischer-Tropsch wax (by coal gasification); vegetable waxes such as carnauba wax, Japan wax, Bayberry wax, rice wax, sugar cane wax, candelilla wax, tallow, and jojoba oil; animal wax such as beeswax, Shellac wax, Spermaceti wax, whale wax, Chinese wax, and lanolin; ester wax; saturated fatty acid amides wax such as capronamide, caprylamide, pelargonic amide, capric amide, laurylamide, tridecanoic amide, myristylamide, stearamide, behenic amide, and ethylene-bisstearamide; unsaturated fatty acid amides wax such as caproleic amide, myristoleic amide, oleamide, elaidic amide, linoleic amide, erucamide, ricinoleic amide, and linolenic amide; mineral waxes such as montan wax, ozokerite, ceresin, and lignite wax; synthetic waxes such as polytetrafluoroethylene wax, Akura wax, and distearyl ketone; hydrogenated waxes such as castor wax and opal wax; and modified waxes such as montan wax derivatives, paraffin wax derivatives, and microcrystalline wax derivatives, and combinations thereof.

Based on the total toner weight, the wax or wax mixture comprising the purified polyalkylene wax of this disclosure such as purified Polywax 1000 and/or Polywax 2000 may generally be present in an amount from about 3% to about 20%, although it may be present in greater or lesser amounts.

As an important additive to the toner particles, the silica imparts several advantageous properties to the toner, including, for example, toner flow, tribo enhancement, admix control, improved development and transfer stability and higher toner blocking temperature. For example, silica may improve and control the toner flow properties of the toner. Toner cohesivity can have detrimental effects on toner handling and dispensing. Toners with excessively high cohesion can exhibit “bridging” which prevents fresh toner from being added to the developer mixing system. Conversely, toners with very low cohesion can result in difficulty in controlling toner dispense rates and toner concentration, and can result in excessive dirt in the machine. In addition, in certain applications, toner particles are first developed from a magnetic brush to donor rolls. Toner flow must be such that the electric development fields are sufficient to overcome the toner adhesion to the donor rolls and enable adequate image development to the photoreceptor. Following development to the photoreceptor, the toner particles must also be able to be transferred from the photoreceptor to the substrate.

Suitable silica may be colloidal silica particles, i.e., silica particles having a volume average particle size, for example as measured by any suitable technique such as by using a Coulter Counter, of from about 5 nm to about 200 nm in an aqueous colloidal dispersion. The colloidal silica may contain, for example, about 2% to about 30% solids, and generally from about 2% to about 20% solids.

In an exemplary embodiment, the colloidal silica particles may have a bimodal average particle size distribution. Specifically, the colloidal silica particles comprise a first population of colloidal silica particles having a volume average particle size of from about 5 to about 200 nm, and generally from about 5 nm to about 100 nm, and a second population of colloidal silica particles having a volume average particle size of about 5 to about 200 nm, and generally about 5 to about 100 nm, although the particle size can be outside of these ranges. The first group of colloidal silica particles may comprise, e.g., SNOWTEX OS supplied by Nissan Chemical Industries (about 8 nm), while the second group of colloidal silica particles may comprise, e.g., SNOWTEX OL supplied by Nissan Chemical Industries (about 40 nm).

It is believed that the smaller sized colloidal silica particles are beneficial for toner gloss, while the larger sized colloidal silica particles are beneficial for toner release properties. Therefore the toner release properties and the toner gloss may be controlled by varying the ratio of differently sized colloidal silica particles.

Based on the total toner weight, silica may generally be present in an amount from about 0% to about 20%, including from about 3% to about 15%, and from about 4% to about 10%, although it may be present outside the ranges. In case the silica contains a first group of colloidal silica and a second group of colloidal silica, the first group of colloidal silica particles are present in an amount of from about 0.0% to about 15%, and generally about 0.0% to about 10%, of the total amount of silica; and the second group of colloidal silica particles are present in an amount of from about 0.0% to about 15%, and generally about 0.0% to about 10%, of the total amount of silica.

Various known suitable and effective positive/negative charge enhancing additives can be selected for incorporation into the toner formulation. Examples include quaternary ammonium compounds inclusive of alkyl pyridinium halides; alkyl pyridinium compounds, reference U.S. Pat. No. 4,298,672, the disclosure of which is totally incorporated herein by reference; organic sulfate and sulfonate compositions, U.S. Pat. No. 4,338,390, the disclosure of which is totally incorporated herein by reference; cetyl pyridinium tetrafluoroborates; distearyl dimethyl ammonium methyl sulfate; aluminum salts such as BONTRON E84 or E88 (Hodogaya Chemical); and the like.

Based on the total toner weight, charge enhancing additive may generally be present in an amount from about 0% to about 10%, including from about 1% to about 8%, and from about 2% to about 5%, although it may be present outside the ranges.

In exemplary embodiments, the toner may be prepared by the following procedure

(i) mixing a first portion of a latex with a colorant dispersion, a wax dispersion comprising the purified polyalkylene wax of this disclosure such as purified Polywax 1000 and/or Polywax 2000, and a coagulant, thereby forming a toner slurry;

(ii) heating the toner slurry at or below the glass transition temperature of the latex polymer to form toner sized aggregates;

(iii) adding a second portion of the latex into the toner sized aggregates;

(iv) adjusting the pH of the emulsion system with a base from a pH of about 2.0 to about 2.5, to a pH of about 6.5 to about 7.0 to prevent, or minimize additional particle growth;

(v) heating the toner sized aggregates at a coalescence temperature which is above the glass transition temperature of the latex polymer, thereby coalescing the toner sized aggregates into toner particles;

(vi) optionally treating the toner particles with acidic solutions; and

(vii) optionally isolating, washing, and drying the toner particle.

The resultant product of the toner process can be pulverized by known methods such as milling to form toner particles. The toner particles generally have an average volume particle diameter of about 2 microns to about 25 microns, typically about 3 microns to about 15 microns.

Toners of the disclosure can be used in known electrostatographic imaging methods. Thus, for example, the toners can be charged, e.g., triboelectrically, and applied to an oppositely charged latent image on an imaging member such as a photoreceptor or ionographic receiver. The resultant toner image can then be transferred, either directly or via an intermediate transport member, to a support such as paper or a transparency sheet. The toner image can then be fused to the support by application of heat and/or pressure, for example with a heated fuser roll.

Specific embodiments of the disclosure will now be described in detail. These examples are intended to be illustrative, and the disclosure is not limited to the materials, conditions, or process parameters set forth in these embodiments. All parts and percentages are by weight unless otherwise indicated.

### EXAMPLES

### Example 1

### Purification Process

150-Gallon Polywax 2000 Extraction Process

50 kg Polywax 2000 (Baker-Petrolite) and 292 kg Isopar/Ashpar C (Ashland) were charged into a 150-gallon Cogeim filter-dryer that was fitted with a 0.5 um Gortex filter cloth. Mixing was started at 30 RPM, the filter-dryer was heated to 85° C., and the slurry was mixed for three hours at 85° C. The Ashpar C was filtered off by vacuum, leaving a Polywax 2000 wet cake on the filter cloth. 292 kg fresh Ashpar C was charged into the filter-dryer, and the Polywax 2000 wet cake was reslurried by mixing at 30 RPM. The filter-dryer was again heated to 85° C., the slurry was mixed for three hours at 85° C., and the Ashpar C was filtered off by vacuum. The preceding steps were repeated two more times, for a total of four mixing/filtering steps. The remaining Polywax 2000 wet cake was dried at 85° C. for 18 hours in the filter-dryer, and then discharged as a fine white powder. The powder was comilled through a 60-mesh screen to remove lumps. The final product from this procedure will hereafter be referred to as “purified Polywax 2000”.

### Example 2

DSC Characterization

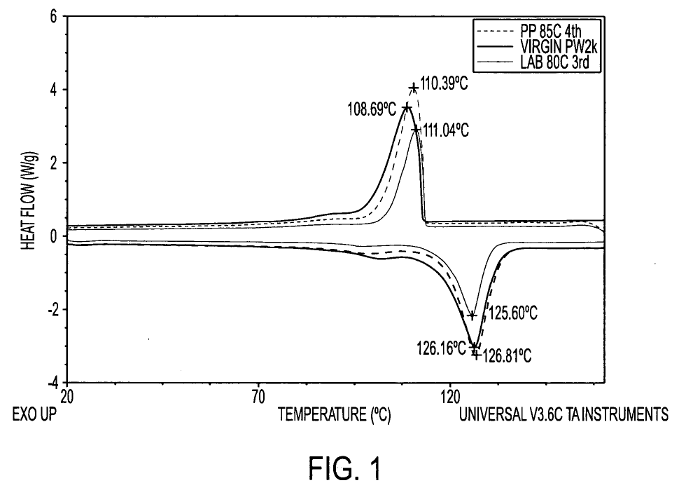

Three different samples are tested by DSC: virgin PW2000, pilot plant purified PW2000 and bench-scale PW2000. The DSC traces are shown below in FIG. 1. The virgin PW2000 (blue) exhibits a broad endothermic event from 90-110° C., which is much bigger than the one of both purified samples (green and brown). In addition, the pilot plant sample (brown) show a more silent feature than the bench scale sample (green). Therefore, the pilot plant sample is more pure than bench scale one.

### Example 3

High Temperature GPC (HT-GPC) Results

Table 1 shows molecular weight characteristics that were measured for three wax samples using a high temperature GPC technique. Table indicates that the purified Polywax 2000 has a higher molecular weight and narrower polydispersity than the unpurified material. Also, analysis of the residue shows that low molecular weight impurities are being removed from the Polywax.

TABLE 1
HTGPC Analysis of Polywax samples

| Samples Description | Mₙ | M_{w} | PDI |
|---|---|---|---|
| Unpurified Polywax 2000 | 1890 | 2746 | 1.45 |
| Purified Polywax 2000 (the 2^{nd} portion) | 2694 | 3418 | 1.27 |
| Residue removed from Polywax 2000 | 1557 | 1999 | 1.28 |
| by extraction process (the 1^{st} portion) | | | |
| Unpurified Polywax 1000 | 1154 | 1243 | 1.08 |
| Purified Polywax 1000 (the 2^{nd} portion) | 1259 | 1325 | 1.05 |
| Residue removed from Polywax 2000 | 840 | 872 | 1.04 |
| by extraction process (the 1^{st} portion) | | | |

### Example 4

HT-GPC Statistical Analysis

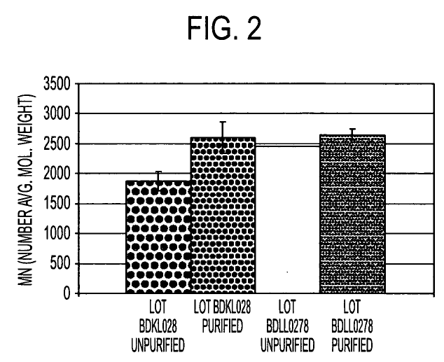

FIG. 2 shows HT-GPC statistical analysis of several purified and unpurified Polywax samples (95% confidence interval is indicated by error bars). The figure indicates that the purified material indeed has a consistently higher number average molecular weight than the unpurified material. Also, the two different lots of unpurified Polywax have significantly different Mn. The purification process thus creates a more consistent supply of wax for processing into the final application.

### Example 5

Electrical Analysis

This example demonstrates the advantage of Purified PW2000 over regular PW2000. Three Gyricon samples made of two different polywax were tested side by side: Unpurified PW2000 and Purified PW2000. Unpurified PW2000 CR dropped after 48 hours, and Purified PW2000 sustained its CR. See Table 2 below.

TABLE 2
Electrical analysis of Gyricon devices made
using unpurified and purified Polywax

AA531, XRCC531

| | | | |
|---|---|---|---|
| 60 V | 80 V | 100 V | 125 V |

Unpurified PW2000

| | | | | |
|---|---|---|---|---|
| Time zero | 2.13 | 3.45 | 4.31 | 4.49 |
| 48 hours | 1.16 | 1.34 | 1.55 | 1.86 |

AA569, XRCC94

| | | | |
|---|---|---|---|
| 60 V | 80 V | 100 V | 125 V |

Purified PW2000

| | | | | |
|---|---|---|---|---|
| Time zero | 3.67 | 3.91 | 3.76 | 3.57 |
| 48 hours | 3.55 | 3.64 | 3.56 | 3.40 |
| 120 hours | 3.26 | 3.60 | 3.60 | 3.50 |

Gyricon devices were shown to have increased contrast ratio value when using the purified materials of this disclosure.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A method of polyalkylene purification comprising:
(i) providing a polyalkylene with a weight average molecular weight M_{w};
(ii) mixing the polyalkylene with a C5-16 alkane;
(iii) dissolving a first portion of the polyalkylene with a weight average molecular weight Mw1<Mw in the C5-16 alkane;
(iv) separating a second portion of the polyalkylene with a weight average molecular weight Mw2>Mw that is insoluble in the C5-16 alkane; and
(v) optionally recovering the first portion of the polyalkylene from its C5-16 alkane solution.

2. The method according to claim 1, in which the polyalkylene comprises linear polyethylene wax.

3. The method according to claim 1, in which the M_{w} ranges from about 1,700 to about 3,700.

4. The method according to claim 3, in which the M_{w} ranges from about 2,200 to about 3,200.

5. The method according to claim 4, in which the M_{w} ranges from about 2,700 to about 2,800.

6. The method according to claim 1, in which M_{w1} ranges from about 0.55M_{w} to about 0.95M_{w}.

7. The method according to claim 6, in which M_{w1} ranges from about 0.70M_{w} to about 0.75M_{w}.

8. The method according to claim 1, in which M_{w2} ranges from about 1.05M_{w} to about 1.45M_{w}.

9. The method according to claim 8, in which M_{w2} ranges from about 1.20M_{w} to about 1.30M_{w}.

10. The method according to claim 1, in which said polyethylene with a weight average molecular weight M_{w} has a polydispersity index PDI; said first portion of the polyethylene has a polydispersity index PDI₁<PDI; and said second portion of the polyethylene has a polydispersity index PDI₂<PDI.

11. The method according to claim 10, in which the PDI ranges from about 1.3 to about 2.0.

12. The method according to claim 10, in which both PDI₁ and PDI₂ are in the range of from about 0.78PDI to about 0.98PDI.

13. The method according to claim 1, in which the C₅₋₁₆ alkane is an isomeric alkane.

14. The method according to claim 1, in which the C₅₋₁₆ alkane is a C₇₋₁₀ isomeric alkane.

15. The method according to claim 1, in which the C₅₋₁₆alkane is selected from one of the following compounds or mixture thereof:

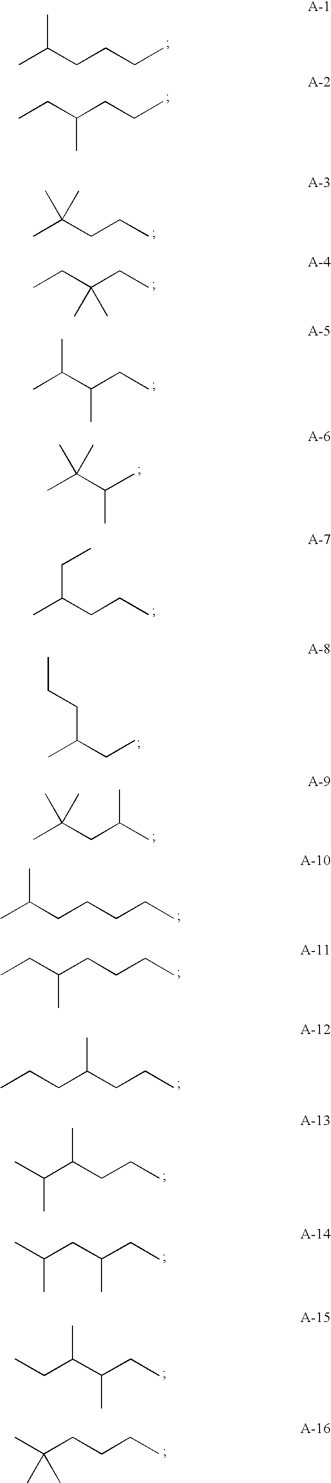

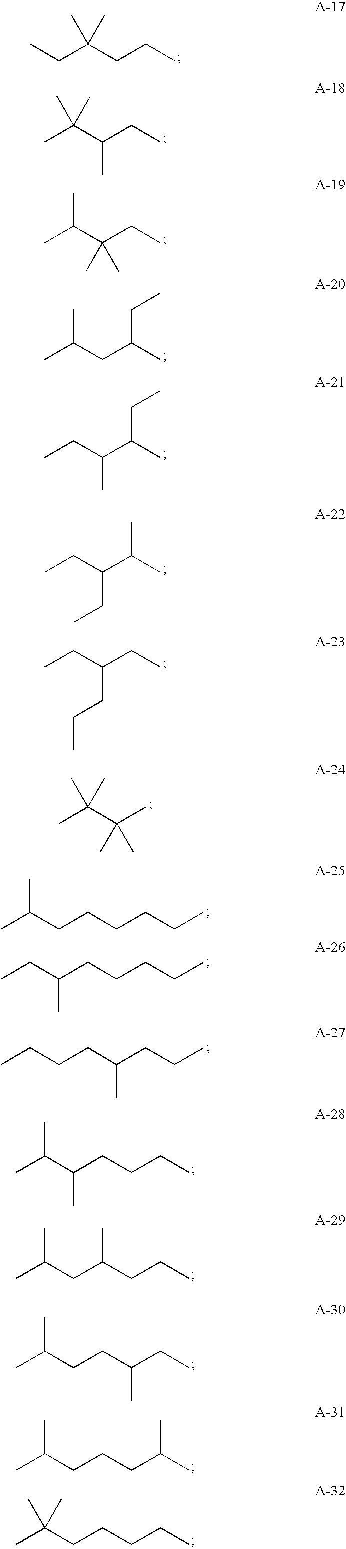

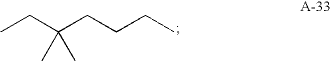

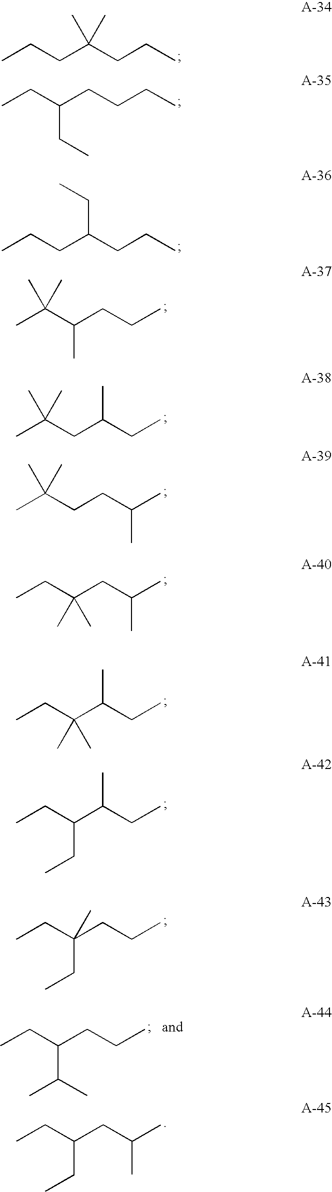

16. The method according to claim 1, in which the steps (ii), (iii) and (iv) are conducted at a temperature of from about 45° C. to about 125° C.

17. The method according to claim 1, in which the weight ratio between the polyethylene with weight average molecular-weight M_{w} and the C₅₋₁₆ alkane is from about 1:2 to about 1:8.

18. The method according to claim 1, a single operation of which can purify at least 30 kg of the polyalkylene with M_{w}.

19. A microencapsulated gyricon bead, which comprises the polyalkylene wax purified by the method of claim 1.

20. The microencapsulated gyricon bead according to claim 19, in which the polyalkylene wax purified by the method of claim 1 is the polyalkylene with a weight average molecular weight M_{w2}.

21. A phase change ink, which comprises a colorant and a carrier comprising the polyalkylene wax purified by the method of claim 1.

22. The phase change ink according to claim 21, in which the polyalkylene wax purified by the method of claim 1 is the polyalkylene with a weight average molecular weight M_{w2}.

23. A toner, which comprises a latex, a colorant dispersion, a coagulant, and a wax dispersion comprising the polyalkylene wax purified by the method of claim 1.

24. The toner according to claim 23, in which the polyalkylene wax purified by the method of claim 1 is the polyalkylene with a weight average molecular weight M_{w2}.
